(19) United States
(12) Patent Application Publication
(10) Pub. No.: US 2022/0244170 A1
(43) Pub. Date: Aug. 4, 2022

(54) STATE DETECTION OF MATERIAL SURFACES OF WEARABLE OBJECTS USING COLOR SENSING

(71) Applicant: 3M INNOVATIVE PROPERTIES COMPANY, St. Paul, MN (US)

(72) Inventors: Nicholas G. Amell, Minneapolis, MN (US); Thaine W. Fuller, Lakeland, MN (US); Jonathan B. Arthur, Hudson, WI (US)

(21) Appl. No.: 17/597,098

(22) PCT Filed: Jun. 19, 2020
(86) PCT No.: PCT/IB2020/055826

§ 371 Date: Dec. 27, 2021

Related U.S. Application Data
(60) Provisional application No. 62/868,490, filed on Jun. 28, 2019.

(51) Int. Cl. G01N 21/25; G01J 3/02; G01J 3/46; G01N 21/88; G01N 21/95; G01L 1/24; D06H 3/08

(52) U.S. Cl. CPC G01N 21/251; G01J 3/0272; G01J 3/463; G01N 2021/8444; G01N 21/95; G01L 1/24; D06H 3/08; G01N 21/8806

(57) ABSTRACT

Systems and methods of state detection of material surfaces of wearable objects using color sensing are provided. A color sensor is used to sense light from material surfaces of wearable objects to obtain color sensing data. State information (e.g., tension, compression, deformation, displacement, level of material wear, etc.) of the material surfaces can be determined based on the obtained color sensing data.

### BACKGROUND

Pressure sensors are widely used in wearable applications to detect tension, compression, or pressure of wearable objects. A common issue with the wearable pressure sensors or other embedded sensors or sensing elements is the degradation of the devices over time of use, including material creep, actual wear to electrical and/or mechanical components, etc.

### SUMMARY

There is a desire to detect the state information (e.g., tension, compression, deformation, displacement, level of material wear, etc.) of a wearable object. The present disclosure provides systems and methods of state detection of material surfaces of wearable objects using color sensing. The material surfaces may include stretchable, compressible or deformable materials that are under various compression or tension states when the wearable object is in use.

In one aspect, the present disclosure describes a method of measuring a state of a wearable object. The method includes providing a wearable object worn by a wearer, the wearable object including one or more material surfaces under compression or tension; providing an optical sensor pack detached from the wearable object, the optical sensor pack including a color sensor configured to sense light from the material surfaces; obtaining, via the optical sensor pack, color sensing data by sensing the light from the material surfaces; and processing, via a processor, the color sensing data from the color sensor to determine state information of the material surfaces. In some cases, the color sensing data are compared to a reference dataset to determine a compression or tension state of the wearable object.

In another aspect, the present disclosure describes a system to detect state information of one or more material surfaces of a wearable object. The system includes a color sensor configured to sense light from the material surfaces of the wearable object to obtain color sensing data; and a computing device functionally connected to the color sensor, the computing device including an analytical module configured to analyze the color sensing data to determine state information of the material surfaces.

Various unexpected results and advantages are obtained in exemplary embodiments of the disclosure. One such advantage of exemplary embodiments of the present disclosure is that state information (e.g., tension, compression, deformation, displacement, level of wear, etc.) of material surfaces of a wearable object in use can be detected in real time by a color sensor detached from the wearable object. Such a color sensing measurement of the material surfaces allows for the quantification of distortion, pressure, wear/damage, placement and motion that may or may not be visible to the human eye.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIG. 1 illustrates a schematic diagram of a system to detect material surfaces of a wearable object using color sensing, according to one embodiment.

FIG. 2 illustrates a flow diagram of a method to detect material surfaces of a wearable object using color sensing, according to one embodiment.

FIG. 3 illustrates a block diagram of a system to detect material surfaces of a wearable object using color sensing, according to one embodiment.

FIG. 4 illustrates an optical sensor pack connected to a mobile device to detect compression socks worn by a wearer, according to one embodiment.

FIG. 5A illustrates a schematic side view of a compression sock with color indices to be detected by color sensing, according to one embodiment.

FIG. 5B illustrates a schematic side view of a compression sock with inherent color encoding to be detected by color sensing, according to one embodiment.

FIG. 5C illustrates a schematic side view of a compression sock with continuous encoding to be detected by color sensing, according to one embodiment.

FIG. 6A illustrates an optical image for a woven material sample at rest.

FIG. 6B illustrates an optical image for the woven material of FIG. 6A under a stretch tension.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

The present disclosure provides systems and methods to detect state information (e.g., tension, compression, deformation, displacement, level of wear, etc.) of material surfaces of wearable objects using color sensing. In some cases, the material surfaces may include stretchable, compressible or deformable materials that are under various compression or tension states when the wearable object is in use. The state information of a wearable object can be determined based on the measured color sensing data from the stretchable, compressible or deformable materials.

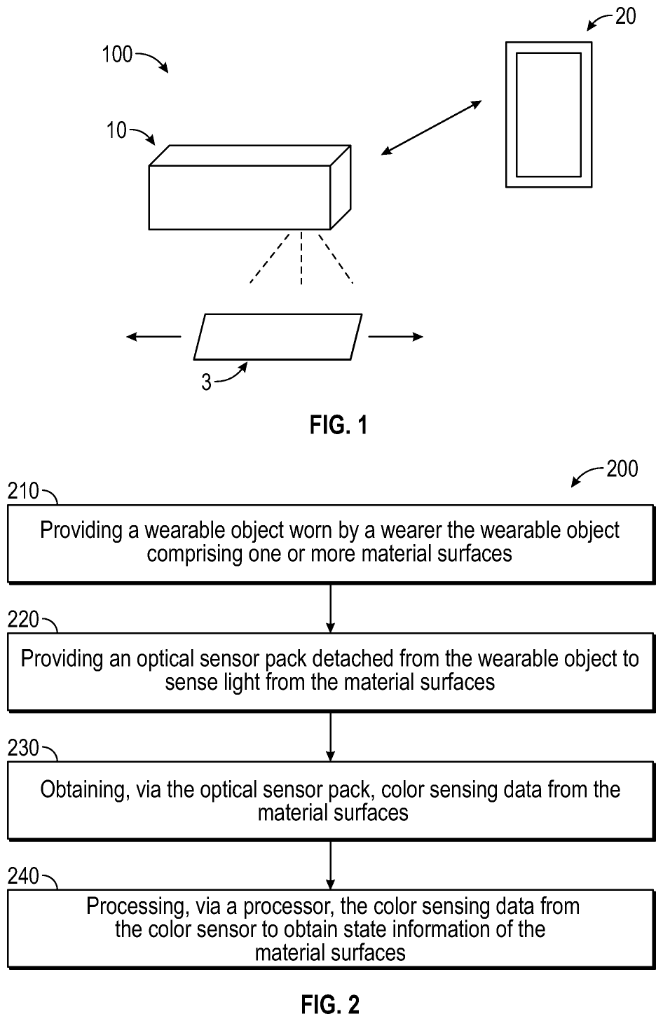

FIG. 1 illustrates a schematic diagram of a system 100 to detect state information of material surfaces of a wearable object 3 using color sensing, according to one embodiment. The system 100 includes an optical sensor 10 configured to sense light reflected from the wearable object 3. In some embodiments, the optical sensor 10 can sense high precision color changes over time for various areas of the wearable object 3. The optical sensor 10 is detached from the wearable object 3 and can be positioned adjacent to the wearable object 3 to determine state information of various targeted areas of the wearable object by detecting, e.g., changes in color within the targeted areas.

The wearable object 3 can include one or more material surfaces which can be under compression or tension when the wearable object 3 is worn by a wearer. Exemplary wearable objects include a wearable brace, a compression sock, a bandage, a flexible wrap, a joint or limb support device, etc. The wearable object 3 can include any suitable stretchable, compressible, or deformable materials such as, for example, a woven material, a nonwoven material (e.g., fibers), a foam material, etc., that is suitable to be worn by a wearer such as, for example, a person, a robot, an animal, or other wearers.

While not wanting to be bound by theory, it is believed that a stretchable, compressible, or deformable material surface, such as a foam or an elastics surface, can change structurally (e.g., a change of porosity size, an exposure of underlying material, a damage to less-flexible materials, etc.) to induce a change of the spectrum and/or optical phase of the reflected light therefrom. For example, woven material surfaces may change in the distance between thread and elastic groupings depending on the direction of distortion, which can also change the spectrum and/or optical phase of the reflected light therefrom. In some cases, a targeted surface area of the wearable object may change its reflected wavelength (e.g., in the form of a material color change) during mechanical stress. Such a material color change can be readable by the system 100 of FIG. 1.

In some embodiments, the spectrum and/or optical phase change of light from the stretchable, compressible, or deformable material surfaces of the wearable object can be derived from the displacement of the pigment in the material of the wearable object. The wearable object can include colored threads or films, and/or material modifications by other material processing techniques at various targeted areas of the wearable object. A color sensor can detect the corresponding spectrum or optical phase change when the wearable object is under a tension, a compression, a deformation, or a displacement.

In some embodiments, the spectrum and/or optical phase change of light from the material surfaces of the wearable object can be derived from the level of material wear of the wearable object. In some embodiments, at least a portion of the deformable material surfaces of the wearable object may change its color as the material wears. The material wear can include, for example, surface abrasion, deterioration of the material structure, etc., which can be detected by the measured color sensing data from the material surfaces.

In some embodiments, the material surfaces of the wearable object can include gradient layers of color. When the layers are changed (e.g., removed or damaged), the induced color change can be detected by the measured color sensing data from the material surfaces. In some embodiments, the material can be designed to express different levels of wear and types of damage through different color changes.

In some embodiments, the material surfaces of the wearable object can include a material having a critical wear warning label embedded in the material. The wear warning label might be a read layer that is not detectable by a color sensor unless being exposed through under certain level of wear.

The system 100 of FIG. 1 can digitally detect and quantify color changes on the surface of unmodified and modified material surfaces through visible or non-visible spectrum optical sensing. The measurement of the color changes for various surface areas of the wearable object 3 allows for the quantification of distortion, pressure, damage, displacement, and motion that may or may not be visible to the human eye.

The optical sensor 10 is functionally connected to a mobile device 20. The mobile device 20 can include a user interface (UI) to receive a user's instruction to obtain, via the optical sensor 10, color sensing data of various target areas of the wearable object 3. The mobile device 20 can further include a computing element, e.g., a processor, to process the color sensing data from the optical sensor 10 to obtain state information of various target areas of the wearable object 3. Exemplary state information include tension, compression, deformation, displacement, level of material wear, etc. The user interface can then present the obtained state information to the user.

FIG. 2 illustrates a flow diagram of a method 200 to detect state information of a wearable object using color sensing, according to one embodiment. At 210, a wearable object is provided to be worn by a wearer. The wearable object includes one or more material surfaces which are under a tension, compression, deformation, or displacement state when worn by the wearer. The method 200 then proceeds to 220.

At 220, an optical sensor pack is provided to sense light from the material surfaces of the wearable object. In some embodiments, the optical sensor pack includes a light source to direct light to the material surfaces of the wearable object. The light source can be, for example, a white-colored LED positioned to illuminate at least a portion of the material surfaces. The optical sensor pack further includes a color sensor to sense the reflected light from the illuminated surface. In some embodiments, the optical sensor pack can be a part of a handheld reader. In some embodiments, a user interface can interact with the user to guide the user to measure various locations of the material surfaces. The method 200 then proceeds to 230.

At 230, the optical sensor pack obtains color sensing data based on the sensed light reflected from the material surfaces. In some embodiments, the color sensing data obtained by the color sensor may include a digital return of color values such as, for example, red, green, blue, and white (RGBW) light sensing values, or red, green, blue (RGB) light sensing values. It is to be understood that color sensing data can be obtained in any suitable color formats. The measurement for each position can be repeated multiple times over a sampling period. Noise in the color sensing data can be eliminated by averaging the obtained color sensing data at each position. The method 200 then proceeds to 240.

At 240, a processor receives the color sensing data from the color sensor and processes the color sensing data to obtain state information of the material surfaces of the wearable object. In some embodiments, the measured color sensing data can be analyzed and compared to a reference dataset to determine a compression or tension state of the wearable object. For example, an analytical module can compare a measured color change for a location to a tension/compression force versus color change curve in a reference dataset. When the measured color change is less than a lower threshold of color change, the analytical module can determine that the tension/compression force at that location is not enough. When the measured color change is greater than an upper threshold of color change, the analytical module can determine that there is too much tension/compression force at that location.

In some embodiments, a reference dataset can include a reference matrix. The reference matrix can include reference color values, e.g., red, green, blue, and white (RGBW) values, measured for various locations on the same material surfaces under different compression or tension states. In some embodiments, a reference dataset may include various curves of tension/compression force versus color change obtained for the corresponding locations of the material surfaces.

In some embodiments, the processor can calibrate the color sensor for the wearable object before use. For example, for a new material surface with unknown properties, color sensing data can be measured at known levels of tension/compression force to develop a calibration matrix providing correspondences between color values and tension or compression state for the new material surface.

In some embodiments, the processor can process the color sensing data from the area to determine a color change of a targeted area of the wearable object after the wearable object is worn by a wearer. In some embodiments, the processor can determine displacement information of the targeted area based on the determined color change.

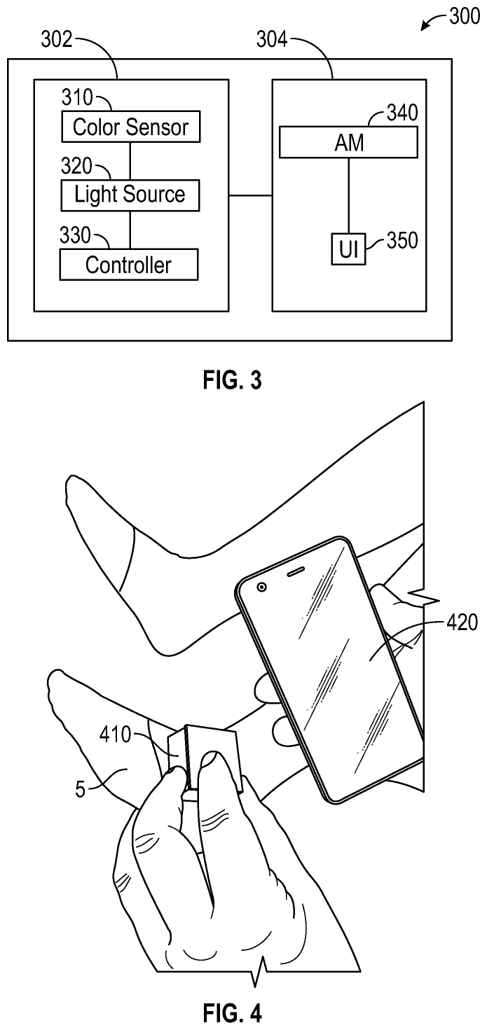

FIG. 3 illustrates a block diagram of a system 300 to detect material surfaces of a wearable object using the method 200 of FIG. 2, according to one embodiment. The system 300 includes a color sensor 310 configured to sense light reflected from material surfaces of a wearable object and obtain color sensing data based on the sensed light. A light source 320 can be integrated with the color sensor 310 to illuminate the material surfaces of the wearable object. In some embodiments, the color sensor 310 and the light source 320 can be integrated as a measurement unit 302, which can be a part of a handheld reader. The measurement unit 302 further includes a controller 330 to allow control of the color sensor 310 and the light source 320. The controller 330 may also provide analysis of the color sensing data from the color sensor 310.

The measurement unit 302 is functionally connected to a computing unit 304. The computing unit 304 includes an analytic module (AM) 340 to process the color sensing data from the measurement unit 302 to determine state information of the material surfaces of the wearable object. The computing unit 304 further includes a user interface (UI) 350 to allow interaction with a user. The computing unit 304 can be integrated into a mobile device such as, for example, a smart phone, or may be integrated into a computer or any other suitable computing device. In some embodiments, the analytic module 340 may operate on a local network or be hosted in a Cloud computing environment. In some embodiments, the user interface 350 can interact with a user via any suitable input/output devices.

The computing unit 304 can include a processor. The processor may include, for example, one or more general-purpose microprocessors, specially designed processors, application specific integrated circuits (ASIC), field programmable gate arrays (FPGA), a collection of discrete logic, and/or any type of processing device capable of executing the techniques described herein. In some embodiments, the processor (or any other processors described herein) may be described as a computing device. In some embodiments, the memory may be configured to store program instructions (e.g., software instructions) that are executed by the processor to carry out the processes or methods described herein. In other embodiments, the processes or methods described herein may be executed by specifically programmed circuitry of the processor. In some embodiments, the processor may thus be configured to execute the techniques for analyzing data related to a fluid network described herein. The processor (or any other processors described herein) may include one or more processors.

In some embodiments, the analytic module 340 can compare the obtained color sensing data to a reference dataset to determine a compression or tension state of the wearable object. The reference datasets for the material surfaces of a wearable object can be stored in a memory to which the analytic module 340 can access.

In some embodiments, the analytic module 340 can compare the obtained color sensing data to a deformation percentage matrix as a reference dataset for one or more deformable material surfaces of the wearable object to be detected. The deformation percentage matrix can include rows of color values for each position of the material surfaces. The rows of color values may correspond to different deformation state of the corresponding position. The analytic module 340 can match the color sensing data to the closest row of color values in the matrix for that position. The following Table 1 illustrates an exemplary deformation matrix for Positions 1, 2 and 3 of the material surface of a wearable object. For each position, there are arrows of measured RGBW values corresponding to different deformation state. Take Position 1 for example. The first row of RGBW values (3963, 989, 1630, 999) corresponds to a state having low compression; the second row of RGBW values (3960, 988, 1630, 980) corresponds to a state having proper compression; and the third row of RGBW values (3960, 987, 1631, 975) corresponds to a state having too high compression.

TABLE 1

| | RGBW values |
|---|---|
| Position 1 | 3963, 989, 1630, 999 |
| | 3960, 988, 1630, 980 |
| | 3960, 987, 1631, 975 |
| Position 2 | 3841, 1139, 1716, 902 |
| | 3841, 1137, 1712, 850 |
| | 3840, 1135, 1710, 825 |
| Position 3 | 5606, 2950, 1682, 920 |
| | 5606, 2925, 1678, 915 |
| | 5600, 2920, 1670, 902 |

In some embodiments, the analytic module 340 can first determine the location of the measured material surface (e.g., Position 1, 2 or 3 in Table 1) by matching the measured color sensing data to the closet range of reference color values of a location. For example, the analytic module 340 determines that a color reading (3961, 987, 1630, 982) for a location best matches the color range of Position 1, then the analytic module 340 can determine the measured location to be Position 1. With the determined location (e.g., at Position 1), the analytic module 340 can match the measured color values to the nearest row of reference values for that position and to determine the corresponding deformation state. For example, the measured color values (3961, 987, 1630, 982) for Position 1 best matches (3960, 988, 1630, 980), which corresponds to a proper compression.

FIG. 4 illustrates an optical sensor pack 410 connected to a mobile device 420 to detect compression socks 5 worn by a wearer, according to one embodiment. The optical sensor pack 410 can include a light source such as the light source 320 of FIG. 3 to illuminate various targeted areas of the compression socks 5. The optical sensor pack 410 can further include a color sensor such as the color sensor 310 of FIG. 3 to sense light reflected from the illuminated areas of the compression socks 5. The optical sensor pack 410 is designed as a handholdable reader to be positioned in proximate to the wearable object, e.g., the compression socks 5 in the embodiment of FIG. 4. The optical sensor pack 410 is functionally connected to the mobile device 420. The mobile device 420 can include a computing unit such as the computing unit 304 of FIG. 3.

The mobile device 420 can run, via the computing unit, a mobile application to guide a user to control the optical sensor pack 410 to detect the wearable object. In some embodiments, the mobile application can guide the user to take measurement by progressing along the wearable object, for example, from the top of the cuff down to the toe, with the optical sensor pack 410 taking multiple repeated measures during the process. The mobile application can provide instructions as to the position and speed of the optical sensor pack 410 with respect to the wearable object.

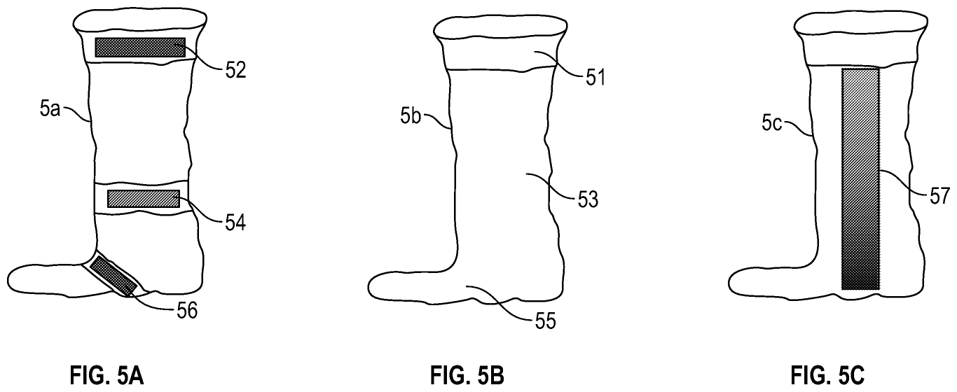

FIG. 5A illustrates a schematic side view of a compression sock 5a with various color indices to be detected by a detecting system described herein, according to one embodiment. The compression sock 5a includes one or more color indices disposed at various locations of the material surface of the compression sock 5a. In the depicted embodiment of FIG. 5A, the compression sock 5a includes a first color index 52 (e.g., blue) located at position 1 (e.g., at the upper calf), a second color index 54 (e.g., red) located at position 2 (e.g., at the ankle), and third color index 56 (e.g., black) at position 3 (e.g., at the foot).

In some embodiments, a mobile application can instruct a user to move an optical sensor pack between the positions by the leg description and/or the detected color indices. The mobile application can further indicate whether the optical sensor pack is placed at the correct location based on an analysis of the measured color sensing data. For example, when an analytic module (e.g., analytic module 340 of FIG. 3) determines that the color reading range from the color sensor falls into a red range, the mobile application can indicate that the optical sensor pack is at position 2 (e.g., at the ankle) where the second color index 54 (e.g., red) is located.

In some embodiments, a color index described herein may include, for example, color fibers woven into the deformable material surface of a wearable object. In some embodiments, a color index described herein may include, for example, a topically colored area of a wearable object. In some embodiments, a color index described herein may include, for example, multiple woven layers of different color that is responsive to visible or non-visible light. The different layers may contribute to a color change upon a state change of the surface material, e.g., when a mechanical stress is applied to the material. In some embodiments, a color index may include one or more surface coatings such as, for example, a coating of paint, pigment, dye, etc., on the surface of the material. Such a surface coating may contribute singularly or in combination with woven layers to the color change. In some embodiments, a color index may include one or more back coatings visible to a color sensor described herein.

FIG. 5B illustrates a schematic side view of a compression sock 5b without color encoding to be detected by a detecting system described herein, according to one embodiment. The compression sock 5b includes no color indices such as color indices 52, 54 and 56 in FIG. 5A. Instead, a reference dataset can be predetermined by building correspondences between the measured color sensing data and stretch/tension/compression forces at various locations (e.g., upper calf 51, ankle 53, foot 55, etc.) of the wearable object (e.g., the compression sock 5b). A user can be instructed, for example, by a mobile application, to move a color sensor between surface positions of the compression sock 5b while the mobile application can indicate the respective locations (e.g., upper calf 51, ankle 53, foot 55, etc.) by comparing the measured color sensing data to the reference dataset. The mobile application can further indicate the state of the material at various locations based on the analysis of the measured color sensing data. For example, an analytic module (e.g., analytic module 340 of FIG. 3) can process the color sensing data and compare it to the reference dataset to determine whether the various locations (e.g., upper calf, ankle, foot, etc.) of the compression sock 5b are under good compression, not enough compression, or too much compression. The reference dataset can be, for example, the deformation matrix such as shown in Table 1 above.

FIG. 5C illustrates a schematic side view of a compression sock 5c with inherent encoding to be detected by a detecting system described herein, according to one embodiment. In this case, a predetermined reference dataset is not necessary. Instead, the material surface of the wearable object may be provided with a known distribution of material color distribution. For example, as shown in the embodiment of FIG. 5C, the compression sock 5c is provided with a continuous color gradient 57, which may be printed on or embedded into the material surface. The color gradient 57 has one or more color values increasing from the upper calf to the ankle. A color sensor can detect the continuous color gradient 57 to determine the associated position/location on the compression sock 5c.

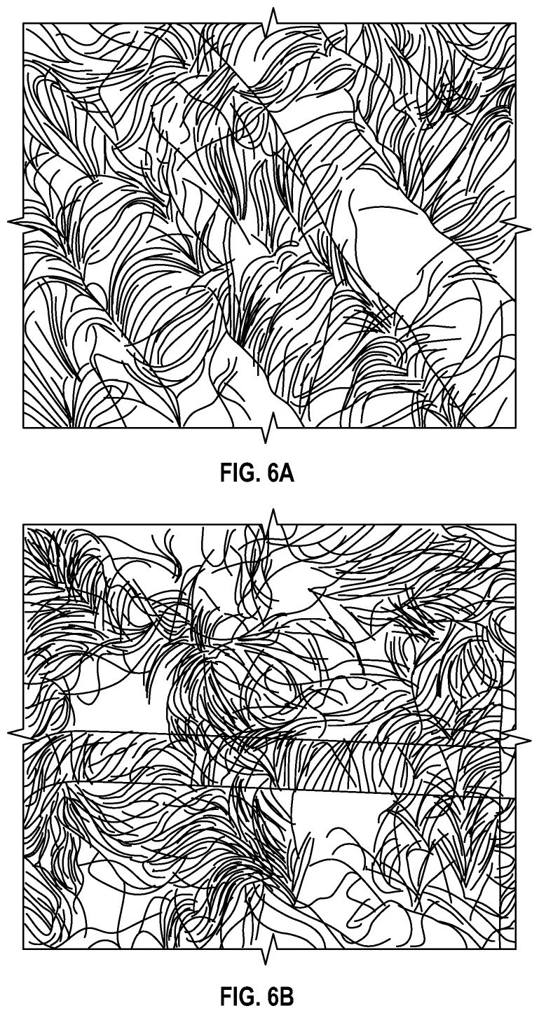

FIG. 6A-B illustrate optical images for a woven material sample at rest (FIG. 6A) and under a stretch tension (FIG. 6B). The stretch tension can be detected by a state detection system and method described herein. Table 2 below lists the detected color values for the same woven material sample under different stretch states. In some embodiments, when the material surface under a tension, a strain, a stretch, or a compression, the surface material (e.g., fibers) may separate from each other, revealing the surface of the object underneath of which it is wrapping or on top of, which can change the color reading, as shown by the RGBW values in Table 2.

TABLE 2

| Strain Displacement | | Strain 1 | Strain 2 |
|---|---|---|---|
| (mm) | No tension | (5 mm) | (15 mm) |
| Clear* | 690 | 960 | 1200 |
| Red | 240 | 330 | 405 |
| Green | 230 | 325 | 405 |
| Blue | 210 | 290 | 360 |

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

### Listing of Exemplary Embodiments

Exemplary embodiments are listed below. It is to be understood that any one of embodiments 1-17 and 18-23 can be combined.

Embodiment 1 is a method of measuring a state of a wearable object, the method comprising:

providing a wearable object worn by a wearer, the wearable object comprising one or more material surfaces under compression or tension;

providing an optical sensor pack detached from the wearable object, the optical sensor pack comprising a color sensor configured to sense light from the material surfaces;

obtaining, via the optical sensor pack, color sensing data by sensing the light from the material surfaces; and

processing, via a processor, the color sensing data from the color sensor to obtain state information of the material surfaces.

Embodiment 2 is the method of embodiment 1, wherein processing the color sensing data comprises comparing the color sensing data to a reference dataset to determine a compression or tension state of the wearable object.
Embodiment 3 is the method of embodiment 2, wherein the reference dataset comprises a deformation percentage matrix.
Embodiment 4 is the method of embodiment 3, wherein the deformation percentage matrix comprises color reference values for the one or more material surfaces under different compression or tension states.
Embodiment 5 is the method of any one of embodiments 1-4, wherein the color sensing data from the color sensor comprises a digital return of light color sensing values.
Embodiment 6 is the method of any one of embodiments 1-5, wherein the optical sensor pack further comprises a light source configured to illuminate the material surfaces.
Embodiment 7 is the method of any one of embodiments 1-6, wherein the one or more material surfaces comprise one or more of stretchable, compressible or deformable materials.
Embodiment 8 is the method of any one of embodiments 1-7, further comprising calibrating the color sensor for the wearable object before use.
Embodiment 9 is the method of any one of embodiments 1-8, further comprising predetermining a reference dataset for the material surfaces by building correspondences between the color sensing data and the respective states of the material surfaces.
Embodiment 10 is the method of any one of embodiments 1-9, further comprising eliminating, via the processor, noise in the color sensing data by averaging the obtained color sensing data at a position of the wearable object.
Embodiment 11 is the method of any one of embodiments 1-10, further comprising providing one or more color indices to the material surfaces.
Embodiment 12 is the method of embodiment 11, further comprising processing, via the processor, the color sensing data from the one or more color indices to determine location information of the material surfaces.
Embodiment 13 is the method of embodiment 12, further comprising determining, via the processor, displacement information of the material surfaces based on the determined color change.
Embodiment 14 is the method of embodiment 13, wherein the one or more color indices include color fibers woven into the material surfaces.
Embodiment 15 is the method of embodiment 13 or 14, wherein the one or more color indices include a plurality of woven layers of different colors.
Embodiment 16 is the method of any one of embodiments 1-15, wherein the one or more color indices include one or more surface or back coatings.
Embodiment 17 is the method of any one of embodiments 1-16, wherein processing the color sensing data comprises determining a material color change to determine a level of material wear or damage.
Embodiment 18 is a system to detect state of one or more material surfaces of a wearable object, the system comprising:

a color sensor configured to sense light from the material surfaces of the wearable object to obtain color sensing data; and

a computing device functionally connected to the color sensor, the computing device comprising an analytical module configured to analyze the color sensing data to determine state information of the material surfaces.

Embodiment 19 is the system of embodiment 18, further comprising a light source configured to illuminate the material surfaces.
Embodiment 20 is the system of embodiment 19, wherein the optical sensor and the light source are a part of a handheld reader.
Embodiment 21 is the system of any one of embodiments 18-20, wherein the computing device further comprises a user interface to interact with a user and present the state information of the material surfaces to the user.
Embodiment 22 is the system of any one of embodiments 18-21, wherein the analytical module is further configured to compare the color sensing data to a reference dataset to determine a compression or tension state of the wearable object.
Embodiment 23 is the system of any one of embodiments 18-22, wherein the computing device further comprises a microprocessor to process the color sensing data, and a memory to store the processed data.

Reference throughout this specification to “one embodiment,” “certain embodiments,” “one or more embodiments,” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus, the appearances of the phrases such as “in one or more embodiments,” “in certain embodiments,” “in one embodiment,” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

While the specification has described in detail certain exemplary embodiments, it will be appreciated that those skilled in the art, upon attaining an understanding of the foregoing, may readily conceive of alterations to, variations of, and equivalents to these embodiments. Accordingly, this disclosure is not to be unduly limited to the illustrative embodiments set forth hereinabove. Furthermore, various exemplary embodiments described herein and other embodiments are within the scope of the following claims.

1. A method of measuring a state of a wearable object, the method comprising:
providing a wearable object worn by a wearer, the wearable object comprising one or more material surfaces under compression or tension;
providing an optical sensor pack detached from the wearable object, the optical sensor pack comprising a color sensor configured to sense light from the material surfaces;
obtaining, via the optical sensor pack, color sensing data by sensing the light from the material surfaces; and
processing, via a processor, the color sensing data from the color sensor to obtain state information of the material surfaces.

2. The method of claim 1, wherein processing the color sensing data comprises comparing the color sensing data to a reference dataset to determine a compression or tension state of the wearable object.

3. The method of claim 2, wherein the reference dataset comprises a reference matrix.

4. The method of claim 3, wherein the reference matrix comprises rows of color reference values for the one or more material surfaces under different compression or tension states.

5. The method of claim 1, wherein the color sensing data from the color sensor comprises RGB values.

6. The method of claim 1, wherein the optical sensor pack further comprises a light source configured to illuminate the material surfaces.

7. The method of claim 1, wherein the one or more material surfaces comprise one or more of stretchable, compressible or deformable materials.

8. The method of claim 1, further comprising calibrating the color sensor for the wearable object before use.

9. The method of claim 1, further comprising predetermining a reference dataset for the material surfaces by building correspondences between the color sensing data and the respective states of the material surfaces.

10. The method of claim 1, further comprising providing one or more color indices to the material surfaces.

11. The method of claim 10, further comprising processing, via the processor, the color sensing data from the one or more color indices to determine location information of the material surfaces.

12. The method of claim 10, wherein the one or more color indices include color fibers woven into the material surfaces.

13. The method of claim 10, wherein the one or more color indices include a plurality of woven layers of different colors.

14. The method of claim 10, wherein the one or more color indices include one or more surface or back coatings.

15. The method of claim 1, wherein processing the color sensing data comprises determining a material color change to determine a level of material wear or damage.

16. A system to detect state of one or more material surfaces of a wearable object, the system comprising:
a color sensor configured to sense light from the material surfaces of the wearable object to obtain color sensing data; and
a computing device functionally connected to the color sensor, the computing device comprising an analytical module configured to analyze the color sensing data to determine state information of the material surfaces.

17. The system of claim 16, further comprising a light source configured to illuminate the material surfaces.

18. The system of claim 17, wherein the optical sensor and the light source are a part of a handheld reader.
